(19) United States
(12) Patent Application Publication
(10) Pub. No.: US 2024/0342495 A1
(43) Pub. Date: Oct. 17, 2024

(54) MONITORING AND CLASSIFICATION OF CARDIAC ARREST RHYTHM

(71) Applicant: Regents of the University of Minnesota, Minneapolis, MN (US)

(72) Inventors: Anthony Robert PRISCO, Minneapolis, MN (US); Alex DEAKYNE, Minneapolis, MN (US); Marinos KOSMOPOULOS, Minneapolis, MN (US); David BENDITT, Minneapolis, MN (US); Pierre SEBASTIAN, Minneapolis, MN (US); Demetris YANNOPOULOS, Minneapolis, MN (US)

(21) Appl. No.: 18/579,604

(22) PCT Filed: Jul. 13, 2022
(86) PCT No.: PCT/US2022/036991

§ 371 Date: Jan. 16, 2024

Related U.S. Application Data
(60) Provisional application No. 63/203,216, filed on Jul. 13, 2021.

(51) Int. Cl. A61N 1/39

(52) U.S. Cl. CPC A61N 1/39044; A61N 1/3993

(57) ABSTRACT

Embodiments comprise an electrocardiograph device for detecting electrical signals of a ventricular fibrillation event in a patient and producing ECG data, a classifier, and a display device. The classifier comprises a memory and a processor, the memory storing a model and one or more parameters of the model. The memory further stores instructions that when executed by the processor the processor to receive the ECG data, and generate a determination indicating whether the ventricular fibrillation event has been caused by heart muscle ischemia based on the model, the parameters of the model. The display device is configured to output the determination, which can also be communicated to follow-on provider systems. The determination can also indicate a predicted coronary perfusion pressure.

### CROSS-REFERENCE TO RELATED APPLICATION

This application claims priority from U.S. Provisional Application Ser. No. 62/203,216 filed on Jul. 13, 2022, the disclosure of which is hereby incorporated by reference in its entirety.

### FEDERALLY SPONSORED RESEARCH

This invention was made with U.S. government support under HL122323 and HL142696 awarded by the National Institutes of Health. The U.S. government has certain rights in the invention.

### TECHNICAL FIELD

Embodiments of the present disclosure relate generally to the field of cardiovascular medicine and more particularly to systems and methods for monitoring and classifying heart rhythms.

### BACKGROUND

Sudden cardiac arrest is usually the result of a fast, erratic, and non-perfusing heart rhythm, commonly ventricular fibrillation (VF). In the United States, nearly 400,000 patients suffer out-of-hospital cardiac arrest (OHCA) each year. Survival rates in this population are abysmal with only about 10% of these cases surviving until hospital discharge. In patients with prolonged cardiopulmonary resuscitation (CPR), the survival rate drops to nearly 0%. The primary goal of bystander CPR is to temporarily circulate blood in a patient allowing oxygen delivery preventing organ specific damage until spontaneous circulation can be restored (via defibrillation), or a more durable solution can be implemented, including initiation of a mechanical CPR device, or placing a patient on cardiopulmonary bypass (referred to as extracorporeal cardiopulmonary resuscitation; i.e. ECPR). The organ most sensitive to hypoxia is the brain and anoxic injury is the most common mechanism of death in this population.

As such, in sudden cardiac arrest, resuscitation of a patient must occur rapidly, otherwise the patient will die. Resuscitation requires reversal of the underlying reason the patient suffered a sudden cardiac arrest, the longer this takes the lower the likelihood of patient survival. Often it takes several minutes to reverse VF, and in this instance, cardiopulmonary resuscitation (CPR) with chest compressions must be performed either until VF is reversed, or the patient is placed on cardiopulmonary bypass.

Sudden cardiac arrest has many etiologies. One of the most common reasons is inadequacy of oxygenated blood flow to heart muscle (i.e., ischemia). Ischemia may be due to for insufficient blood flow at certain times such as during physical or emotional stress, or the acute occlusion/narrowing of one of the main arteries of the heart (otherwise known as a myocardial infarction or “heart attack”). In such cases the opening of a narrowed/occluded artery can be lifesaving. However, in many cases the cardiac arrest is not associated with narrowing or occlusion of an artery (e.g., non-ischemic heart disease or primary electrical heart disease). In such cases, searching for a blocked artery is unnecessary, costly, and takes time away from other treatments.

However, it is often difficult to terminate VF and restore stable heart rhythm and blood pressure in the presence of ongoing myocardial ischemia in a large proportion of sudden cardiac arrest victims. Often, it is impossible to terminate VF (which is an otherwise fatal arrhythmia) without providing circulatory support. Circulatory support can be provided by, for example, extracorporeal membrane oxygenation (ECMO), cardiopulmonary bypass, and/or opening the arteries and relieving the heart muscle ischemia.

Successful restoration of spontaneous circulation (ROSC) requires reversing the underlying cause of the cardiac arrest. A significant portion of OHCA patients who present with ventricular fibrillation (VF) have a significant burden of coronary artery disease (CAD). An acute myocardial infarction (MI) is a frequent cause of the OHCA (VF). As conventional CPR does not reverse an acute MI, CPR often must be done until a patient can be brought to a cardiac catheterization laboratory where an angioplasty can be performed opening a blocked artery.

Currently, at the time of ongoing resuscitation, only after VF is reversed will myocardial ischemia/infarction be diagnosed. Once this is done, if a patient is deemed stable enough, the patient will be taken to a cardiac catheterization laboratory, where the blocked artery or arteries can be identified and potentially addressed. Typically, this first requires transport to a hospital emergency department.

The standard of care for victims of cardiac arrest currently utilizes trained community-based emergency medical service (EMS) providers who are summoned when a suspected cardiac arrest is identified by a bystander. EMS providers use a combination of electrical shocks, anti-arrhythmic therapy, and inotropes (drugs that help increase the force of heart muscle contraction) in an attempt to terminate VF, and restore both a stable heart rhythm, and blood pressure. None of these techniques can relieve a narrowed or blocked artery, however. Furthermore, the general standard of medical care often does not require bringing all patients immediately to a catheterization laboratory or other facility where procedures to locate and alleviate blockages can occur. Factors such as distance from a capable facility, patient stability, traffic and other factors may impact treatment feasibility. Though patients suffering from myocardial ischemia would be highest priority, if they could be identified.

CPR can be either performed manually (by pressing on a patient's chest) or automatically, using a device that is wrapped around a patient's chest which compresses the patient's chest according to guidelines issued by the American Heart Association. Unfortunately, guideline driven CPR is a “one-size-fits-all” approach and compressions are not optimized for a specific patient. Studies have demonstrated that varying compression depth and recoil amount can improve CPR quality, however, this requires invasive monitoring of cardiac perfusion pressure (CPP), which is not logistically feasible in the real world. Further, current guidelines recommend that chest compression are stopped every two minutes in order to assess whether a defibrillation attempt is indicated, which can result in organ hypoperfusion.

Neurologically intact survival improves when the length of time between cardiac arrest and stable perfusion is achieved (via ECPR or a patient's native cardiac function) is decreased. Using the current standard of care for treatment of OHCA, transport to a cardiac catheterization laboratory by EMTs may be significantly delayed as an underlying MI could be preventing ROSC, which in turn prevents diagnosis of the etiology of the cardiac arrest. Simply put, an acute MI cannot be diagnosed until VF is terminated, which is difficult without first treating the MI.

Consequently, it is valuable as soon as possible to distinguish between cardiac arrest associated with ischemia from those due to non-ischemic causes, to optimize CPR delivery to improve cardiac perfusion pressure, and to determine optimal timing of defibrillation therapy during CPR delivery.

### SUMMARY

Embodiments of the present disclosure provide systems and methods for determining the cause of a cardiac arrest.

Embodiments improve outcomes for victims of OHCA by decreasing the time required to return spontaneous circulation, and to increase the quality of CPR done while the patient is in VF. Embodiments can diagnose an acute myocardial infarction in the absence of restoration of spontaneous circulation (while the patient is still in VF), and to optimize CPR (both perfusion pressure, and timing of defibrillation) through closed-loop feedback analysis of a patients underlying arrhythmia (VF). Embodiments can employ various machine learning (ML) analyses of an arrhythmia including Ensemble Classifiers (EnCs), Convolutional Neural Networks (CNNs), and Bayesian Recurrent Neural Networks (BRNNs). Embodiments can infer hemodynamic measures such as cardiac profusion pressure (CPP) non-invasively, therefore reducing the need for invasive measurements. Embodiments can further predict the likelihood of defibrillation success prior to pausing CPR.

In a first aspect of the present disclosure, a cardiac arrest classification and treatment system comprises an electrocardiograph (ECG) device for receiving ECG data produced from electrical signals of a ventricular fibrillation event detected in a patient, a processor, and a memory storing instructions that when executed by the processor cause the processor to implement an ischemia classifier, a coronary perfusion pressure (CPP) classifier, and a therapy determinator.

The ischemia classifier comprises an ischemia model and one or more parameters of the ischemia model, and is configured to generate an ischemia determination based on the ischemia model, the parameters of the ischemia model, and the ECG data. The ischemia determination indicates whether the ventricular fibrillation event has been caused by heart muscle ischemia.

The CPP classifier comprises a CPP model and one or more parameters of the CPP model, and is configured to generate a CPP determination based on the CPP model, the parameters of the CPP model, and the ECG data. The CPP determination indicates a predicted CPP of the patient.

The therapy determinator can be configured to direct delivery of defibrillation therapy to the patient in response to an ischemia determination indicating that the ventricular fibrillation event has been caused by heart muscle ischemia and direct delivery of CPR compressions to the patient based on the CPP determination.

In embodiments, the system further comprises a cardioversion success classifier configured to generate a likelihood of cardioversion success based on the ECG data. The therapy determinator can be further configured to select a time window during which to direct delivery of defibrillation therapy based on the likelihood of cardioversion success, direct a pause in the delivery of CPR compressions to the patient during the time window, and direct the delivery of defibrillation therapy to the patient during the time window.

In embodiments, the likelihood of cardioversion success can be based on the CPP determination. The likelihood of cardioversion success can be determined to be high when the CPP determination is equal to or greater than a threshold, such as 15 mmHg.

In embodiments, the system further includes an output device, and directing the delivery of defibrillation therapy comprises producing an output directing a user to deliver defibrillation therapy.

In embodiments, the system further includes a defibrillation device electrically coupleable to the patient for the delivery of defibrillation therapy and configured to deliver a defibrillation shock to the patient in response to the direction to delivery defibrillation therapy.

In embodiments, the therapy determinator is further configured to determine a rate and pressure of CPR compressions to be delivered to the patient. The delivery of CPR compressions can comprise producing an output directing a user to deliver CPR compressions at the determined rate and pressure. In embodiments the system includes an automated CPR module arrangeable to deliver CPR compressions to the patient at the determined rate and pressure in response to the direction to deliver CPR compressions.

In a second aspect of the present disclosure, a cardiac arrest rhythm classification and treatment system comprises an ECG device for detecting electrical signals of a ventricular fibrillation event in a patient and producing ECG data, an ischemia classifier comprising a memory and a processor, the memory storing an ischemia model and one or more parameters of the ischemia model, and instructions that when executed by the processor cause the processor to generate an ischemia determination based on the ischemia model, the parameters of the ischemia model, and the ECG data, the ischemia determination indicating whether the ventricular fibrillation event has been caused by heart muscle ischemia. The system can include a display device a display device communicatively coupled to the ischemia classifier and configured to output the ischemia determination.

In a third aspect of the present disclosure, a cardiac arrest classification and treatment system comprises an ECG device for receiving ECG data produced from electrical signals of a ventricular fibrillation event detected in a patient, a CPP classifier comprising a memory and a processor, the memory storing a CPP model and one or more parameters of the CPP model, and instructions that when executed by the processor cause the processor to generate a CPP determination based on the CPP model, the parameters of the CPP model, and the ECG data, the CPP determination indicating a predicted coronary perfusion pressure of the patient, and a display device communicatively coupled to the classifier and configured to output the CPP determination.

In a fourth aspect of the present disclosure, a cardiac arrest classification and treatment system comprises an ECG device for detecting electrical signals of a ventricular fibrillation event during which a patient is receiving cardiopulmonary resuscitation (CPR) compressions and producing ECG data, a cardioversion success classifier configured to generate a likelihood of cardioversion success based on the ECG data, and a therapy determinator configured to select a time window during which to direct delivery of defibrillation therapy based on the likelihood of cardioversion success direct a pause in the delivery of CPR compressions to the patient during the time window, and direct delivery of defibrillation therapy to the patient during the time window.

In embodiments, the cardioversion success classifier can be configured to generate the likelihood of cardioversion success by calculating a predicted coronary perfusion pressure of the patient and determining that the likelihood of cardioversion success is high when the predicted coronary perfusion pressure of the patient is equal to or greater than a threshold.

In embodiments, the system can further comprise an automated external defibrillation module and/or an automated cardiopulmonary resuscitation module.

In embodiments, the electrical signals of the ventricular fibrillation event comprise a short epoch, which can be between 2 and 30 seconds, or more specifically 10 seconds.

In a fifth aspect of the present disclosure, a computer-implemented method for classifying the etiology of a cardiac arrest comprises storing, in a memory, an ischemia model and one or more parameters of the ischemia model, receiving, from an electrocardiograph (ECG) device, ECG data representing electrical signals of a ventricular fibrillation event in a patient, and generating an ischemia determination based on the ischemia model, the parameters of the ischemia model, and the ECG data, the ischemia determination indicating whether the ventricular fibrillation event has been caused by heart muscle ischemia.

In embodiments, the method further includes performing a procedure on the patient to relieve heart muscle ischemia when the ischemia determination is that the ventricular fibrillation event has been caused by heart muscle ischemia.

In a sixth aspect of the present disclosure, a computer-implemented method for optimizing the delivery of CPR to a patient includes storing, in a memory communicatively couplable to a processor, a CPP model and one or more parameters of the CPP model, receiving, from an ECG device, ECG data representing electrical signals of a ventricular fibrillation event in a patient, generating, by the processor, a determination based on the CPP model, the parameters of the CPP model, and the ECG data, the determination indicating a predicted coronary perfusion pressure of the patient and delivering one or more CPR compressions to the patient, the timing and pressure of the CPR determined at least partially based on the predicted coronary perfusion pressure.

In a seventh aspect of the present disclosure, a computer-implemented method monitoring and classifying a cardiac arrest rhythm comprises storing, in a memory communicatively couplable to a processor, an ischemia model, parameters of the ischemia model, a coronary perfusion pressure (CPP) model and parameters of the CPP model, receiving, by the processor, ECG data produced from electrical signals of a ventricular fibrillation event detected in a patient, generating, by the processor, an ischemia determination based on the ischemia model, the parameters of the ischemia model, and the ECG data, the ischemia determination indicating whether the ventricular fibrillation event has been caused by heart muscle ischemia, generating, by the processor, a CPP determination based on the CPP model, the parameters of the CPP model, and the ECG data, the CPP determination indicating a predicted CPP of the patient, directing delivery of defibrillation therapy to the patient in response to an ischemia determination indicating that the ventricular fibrillation event has been caused by heart muscle ischemia, directing delivery of CPR compressions to the patient based on the CPP determination.

In embodiments, the method further includes determining a rate and pressure of CPR compressions to be delivered to the patient.

In embodiments, the method further includes generating a likelihood of cardioversion success based on at least one of the ECG data and the CPP determination, selecting a time window during which to direct delivery of defibrillation therapy based on the likelihood of cardioversion success, directing a pause in the delivery of CPR compressions to the patient during the time window, and directing the delivery of defibrillation therapy to the patient during the time window.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures.

FIG. 1 is a schematic diagram depicting an ischemia detection system, according to an embodiment.

FIG. 2 is a graph depicting a ventricular fibrillation waveform, according to an embodiment.

FIG. 3 is a schematic diagram depicting a machine learning network architecture, according to an embodiment.

FIG. 4 is a schematic diagram depicting a machine learning network architecture, according to an embodiment.

FIG. 5A and FIG. 5B are graphs depicting receiver-operator curves produced based on the output of embodiments of the present disclosure.

FIG. 6 is a schematic diagram depicting an ischemia detection system, according to an embodiment.

FIG. 7 is a flowchart depicting a method for classification of the etiology of cardiac arrest, according to an embodiment.

FIG. 8 is a flowchart depicting a method for updating parameters of a model, according to an embodiment.

FIG. 9 is a graph depicting example ECG data and corresponding CPP data that can be provided as training inputs for embodiments.

FIG. 10 is a graph depicting the moving average CPP of the CPP data depicted in FIG. 9.

FIG. 11 is a graph depicting a subset of the data depicted in FIG. 9.

FIG. 12 is a graph depicting a receiver-operator curve produced based on an embodiment.

FIG. 13 is a graph depicting the sensitivity and positive predictive value of the embodiment of FIG. 12.

FIG. 14 is a graph depicting a receiver-operator curve produced based on an embodiment.

FIG. 15 is a graph depicting the sensitivity and positive predictive value of the embodiment of FIG. 14.

FIG. 16 is a graph depicting the actual and predicted values produced by an embodiment.

FIG. 17 is a graph depicting a receiver-operator curve produced by the embodiment of FIG. 16.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

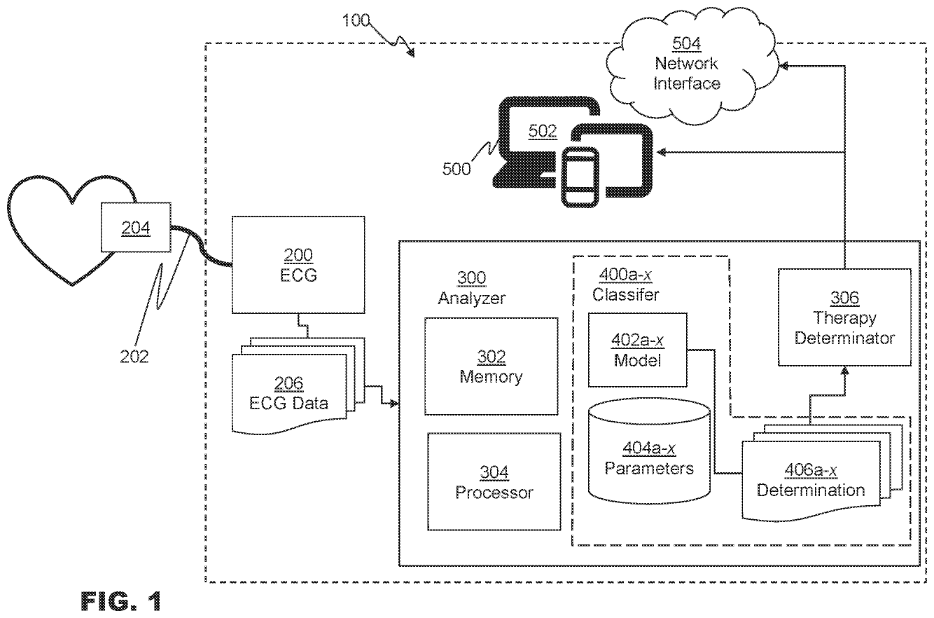

FIG. 1 is a schematic diagram depicting an ischemia detection system, according to an embodiment. System 100 can comprise an electrocardiogram (ECG) sensor 200, analyzer 300 (including one or more classifiers 400a-x), and display device 500.

ECG sensor 200 can comprise one or more leads 202. Each lead 202 can comprise one or more electrodes 204 for placement proximate the body of a patient. Each lead 202 can further comprise electrically conductive, insulated wires, for transmitting electrical energy from electrodes 204 to ECG sensor 200. In an embodiment, ECG sensor 200 comprises a single, bipolar lead 202 with two electrodes 204. In other embodiments, multiple leads 202 with more or fewer electrodes can be used. For example, ECG sensor can comprise six, twelve, or other numbers of leads. Each lead 202 can be unipolar (single electrode), bipolar (two electrode), or include other numbers of electrodes.

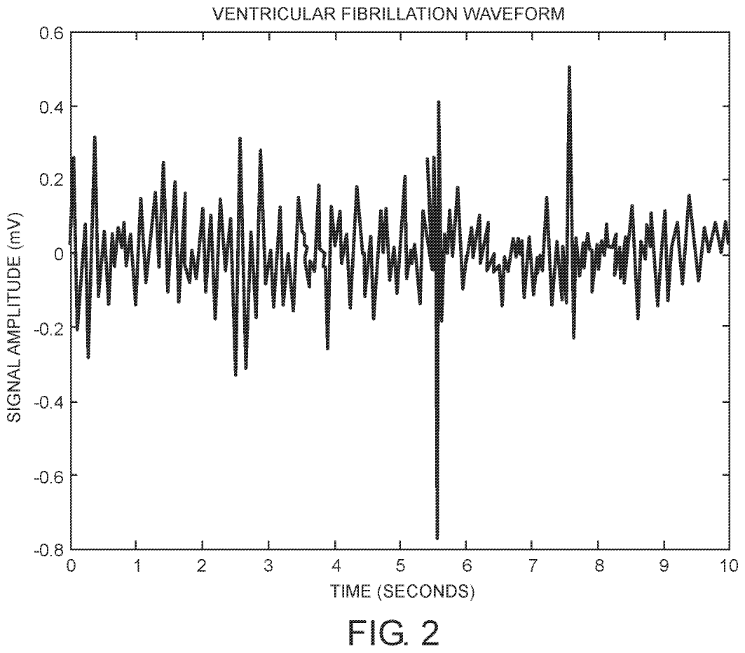

ECG sensor 200 can comprise a voltmeter or other component for measuring the electrical signal between electrodes 204 of lead (or leads) 202. Embodiments can measure one or more aspects of the electrical signal such as voltage, current, capacitance, and the like. FIG. 2 is a graph depicting a waveform as may be recorded by ECG sensor 200. As depicted in FIG. 2, the waveform indicates the amplitude of the electrical potential (in millivolts) over a time a ten second time period (or epoch) of monitoring a patient experiencing ventricular fibrillation, though other time periods, such as any between two seconds and thirty seconds, can be used. ECG sensor 200 can transmit recorded signals to classifier 400 as ECG data 206.

ECG data 206 can comprise raw signal data, such as streams of binary or text data representing discrete measurements of electrical activity. In other embodiments, ECG data 206 can comprise waveform data in an image format, or one or more standard ECG waveform data formats such as Health Level Seven (HL7) Annotated ECG Waveform Data Standard (aECG), Mortara XML (E-Scribe & H-Scribe), GE MUSE XML (Transactional XML), Philips XML (Sierra XML), Schiller XML, AMPS binary ECG, ISHNE, SCP, and/or MIT. In embodiments, ECG data 206 can further comprise non-cardiac patient data, including demographic information of the patient (such as age, sex, gender, which can be provided to various classifiers 400).

Returning now to FIG. 1, analyzer 300 can comprise a computing device including a memory 302 and a processor 304. Memory 302 can provide storage for one or more classifiers 400a-x. Each classifier 400 can comprise one or more machine learning algorithms or models 402 and associated parameters 404. Each model 402 can receive ECG data 206 and produce an associated determination 406.

For example, in an embodiment, analyzer 300 can comprise ischemia classifier 400a, including ischemia model 402a. Ischemia determination 406a can be an indication of whether ECG data 206 represents a signal indicative of heart muscle ischemia, or a signal that is not indicative of heart muscle ischemia. In embodiments, ischemia determination 406a can be a binary result (ischemia or not ischemia). Ischemia determination 406a can also include an indication the result was inconclusive. In yet other embodiments, Ischemia determination 406a can include a likelihood (such as a decimal or percentage chance) that ECG data 206 represents a signal indicative of heart muscle ischemia.

Display device 500 is communicatively coupled to analyzer 300 to receive each determination 406 and output the determination 406 to the user. Display device 500 can comprise a visual display, such as a computer screen or monitor, a digital readout device, light or dial indicators, or the like. Display device 500 can comprise an audio output, such as speakers or headphones. In embodiments, display device 500 can comprise a computing device including user interface 502.

User interface 502 can receive user inputs and provide user outputs regarding configuration of system 100. User interface 502 can comprise a mobile application, web-based application, or any other executable application framework. User interface 502 can reside on, be presented on, or be accessed by any computing devices capable of communicating with the various components of system 100, receiving user input, and presenting output to the user. In embodiments, user interface 502 can reside or be presented on a smartphone, a tablet computer, laptop computer, or desktop computer.

In embodiments, display device 500 can comprise, or be communicatively connected to network interface 504. Determinations 406 or output of therapy determinator 306 can be transmitted via network interface 504 to one or more remote computing systems. In embodiments, network interface 504 can enable system 100 to be communicatively coupled to one or more patient care management systems. For example, each determination 406s can be transmitted from an emergency response location, where system 100 is receiving ECG data 206 to a catherization laboratory, such that users can prepare for arrival of a patient for catheter-based procedures to relieve ischemia.

In embodiments, ischemia model 402a can comprise one or more classification algorithms that can be trained, by iterative modification of parameters 308 to recognize signals that are indicative of heart muscle ischemia.

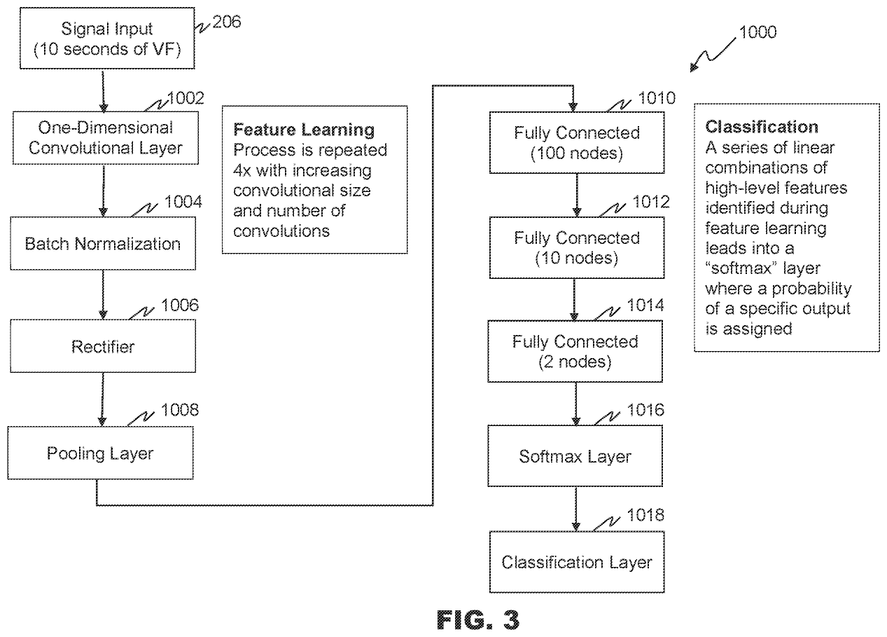

FIG. 3 is a schematic diagram depicting an architecture of a convolutional neural network (CNN) 1000 according to an embodiment. In an embodiment, ischemia model 402a can comprise a CNN such as CNN 1000 for classification of ECG data 206. Convolutional neural networks are a class of artificial neural networks. Convolutional networks can have structures based on a number of types of layers, such as convolutional layers (which compute a dot product between a filter and portions of an input volume), pooling layers (which provide non-linear down-sampling), rectified linear unit (or ReLU) layers, which can increase the nonlinearity of decision functions, and fully connected layers, which can take the input of the previous layers to perform high-level reasoning.

CNN 1000 can comprise a one-dimensional convolutional layer 1002, a batch normalization layer 1004, a rectifier layer 1006, a pooling layer 1008, a first fully connected layer 1010 having one-hundred nodes, a second fully connected layer 1012 having ten nodes, a third fully connected layer 1014 having two nodes, a softmax layer 1016, and a output classification layer 1018. Ischemia model 402aa can receive a signal input, such as 10 seconds of VF ECG data.

In embodiments, the sequence of one-dimensional convolutional layer 1002, batch normalization layer 1004, rectifier layer 1006, and pooling layer 1008 can make up a feature learning sequence, which can be repeated multiple times, with increasing convolutional size and numbers of convolutions. The feature learning sequence can be repeated two, four, six, or any number of times.

In embodiments, the sequence of first fully connected layer 1010, second fully connected layer 1012, third fully connected layer 1014, and softmax layer 1016 can comprise a classification sequence. During the classification sequence, a series of linear combinations of high-level features identified during feature learning leads into softmax layer 1016, where a probability of a specific output can be assigned.

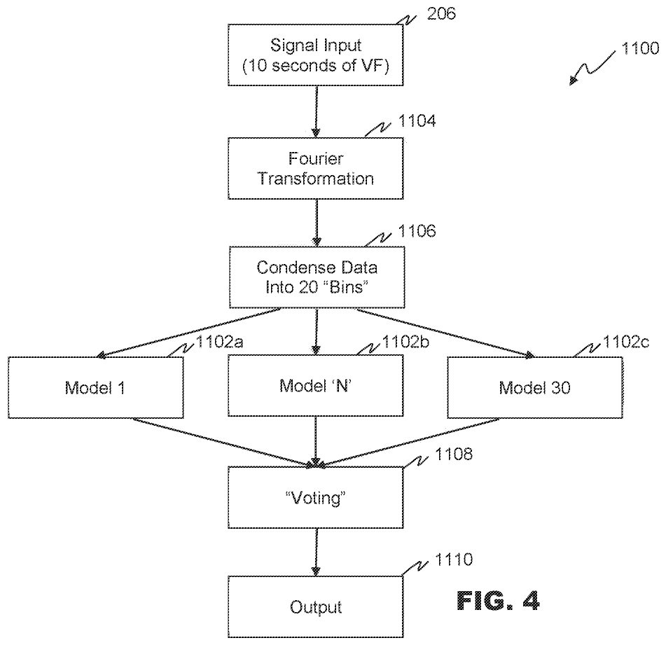

FIG. 4 is a schematic diagram depicting an architecture of an ensemble classifier 1100 according to an embodiment. In an embodiment, ischemia model 402a can comprise an ensemble classifier such as ensemble classifier 1100 for classification of ECG data 206. Ensemble classifier 1100 can comprise a plurality of candidate classifiers 1102. Each candidate classifier 1102 can comprise a classification algorithm or model configured to receive a Fourier transform of ECG data 206 and provide a vote regarding determination 310 regarding whether ECG data 206 is indicative of heart muscle ischemia. Candidate classifiers 1102 can be weak learners.

As depicted in FIG. 4, ECG data 206 can be modified by a Fourier transformation at 1104. The transformed data can be condensed into bins at 1106. In an embodiment, twenty bins are used, though other numbers of bins can be used, such as any number between five bins and one-hundred bins, or any number greater than or equal to ten. The condensed data can then be provided to candidate classifiers 1102. While three candidate classifiers 1102a, 1102b, and 1102c are shown, more or fewer candidate classifiers 1102 can be used. At 1108 the outputs of the classifiers can be combined by voting, and the output classification can be provided at 1110. Voting can comprise calculating a weighted average of the weak learners.

While CNN 1000 and ensemble classifier 1100 are depicted and described, those of ordinary skill in the art will recognize that additional algorithms or classifiers can be used by embodiments.

For evaluation purposes, an experimental data set was created by collecting ECG data monitored from porcine subjects experiencing VF of known etiology. The experimental data was divided into a training data set and a test data set. After designing the architecture for each method, the respective were trained algorithms to recognize whether a cardiac arrest was secondary to an occluded/narrowed artery (ischemia) or not.

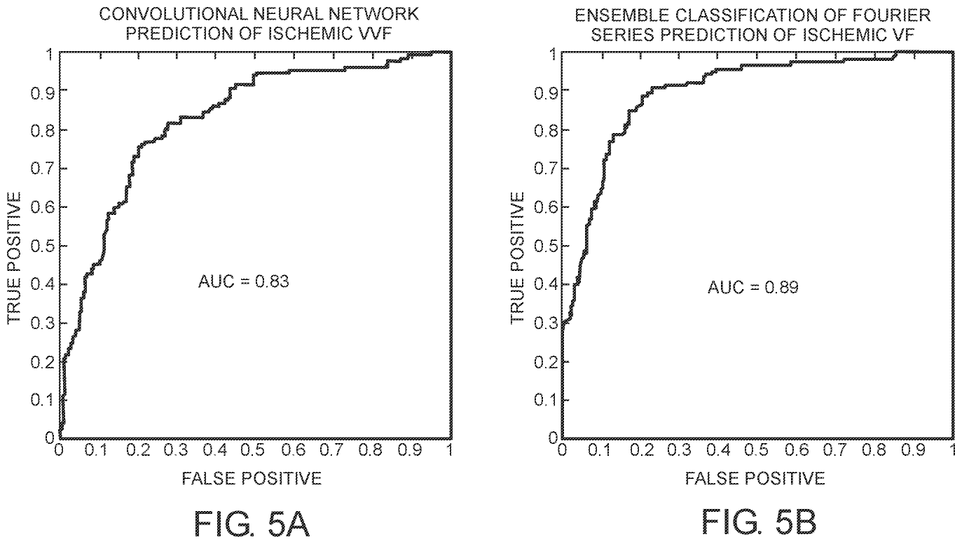

FIGS. 5A and 5B are graphs depicting receiver-operator curves resulting from testing embodiments of system 100. Where the architecture of ischemia model 402a comprised CNN 1000, the area under the curve of false positives against true positives was 0.83. Where the architecture of ischemia model 402a comprised ensemble classifier 1100, the area under the curve was 0.89. To generate the depicted receiver-operator curves, each model was trained with 70% a labeled data set, and the remaining test data was provided to the model.

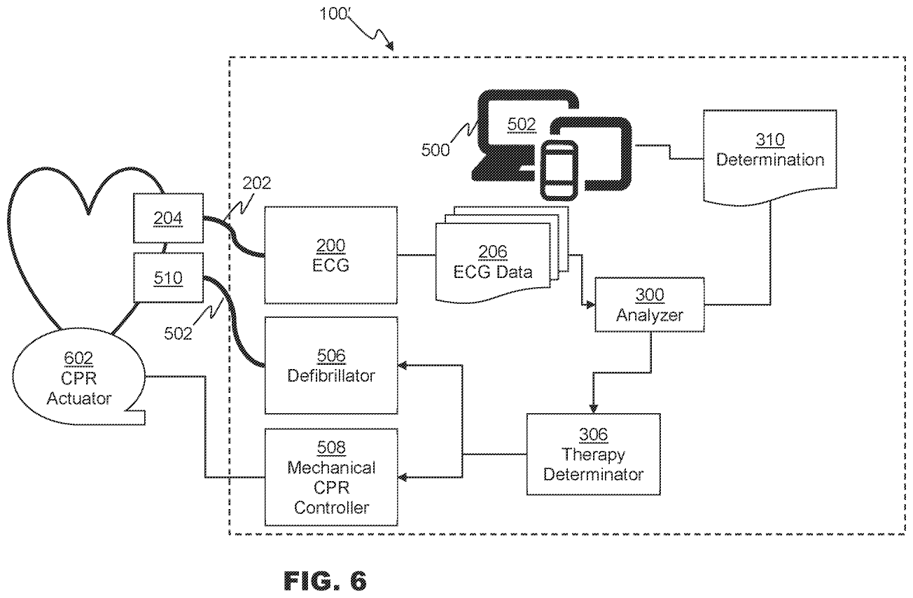

FIG. 6 is a schematic diagram depicting a cardiac treatment system 100′ according to an embodiment. System 100′ can comprise ECG 200, analyzer 300, and display device 500, as described above. System 100′ can further comprise defibrillator 506 and/or mechanical cardiopulmonary resuscitation (CPR) controller 508.

Defibrillator 506 can be an automated external defibrillation device such as those known in the art. Defibrillator 506 can comprise one or more defibrillation leads 508 electrically coupled to electrodes 510 which can be affixed to the body of a patient. In embodiments, electrodes 510 can comprise defibrillation paddles that can be held proximate the body of the patient. Defibrillator 506 can deliver one or more bursts of electrical energy via electrodes 510 to shock the patient in an attempt to terminate a fibrillation event. In embodiments, defibrillator 506 can also receive ECG data 206. ECG data 206 can be used to determine the energy and timing of defibrillation shocks in order to optimize the therapeutic effects of the shocks.

Mechanical CPR controller 508 can be communicatively coupled to CPR actuator 602. CPR actuator 602 can be arranged proximate the chest of the patient to provide automated chest compressions. Mechanical CPR controller 508 can receive ECG data 206 and/or output from therapy determinator 306 in order to optimize the energy and timing of compressions in order to optimize the therapeutic effect of CPR compressions. One method for optimizing CPR delivery is described in Pierre S. Sebastian et al., Closed-loop machine-controlled CPR system optimises haemodynamics duringprolonged CPR, 3 Resuscitation Plus 100021 (2020), https://doi.org/10.1016/j.resplu.2020.100021, though other methods can be used by embodiments.

Integration of with an automatic external defibrillator (AED, a portable defibrillator commonly carried by emergency medical services) can improve patient mortality and reduce costs by reducing the time required to identify a patient appropriate for ischemia relief treatment (i.e., one likely to have a blocked or narrowed heart artery) and have them treated in the cardiac catheterization laboratory. Further, integration of mechanical CPR apparatus can enable continued CPR treatment while the patient is transported and/or prepared for catherization treatment.

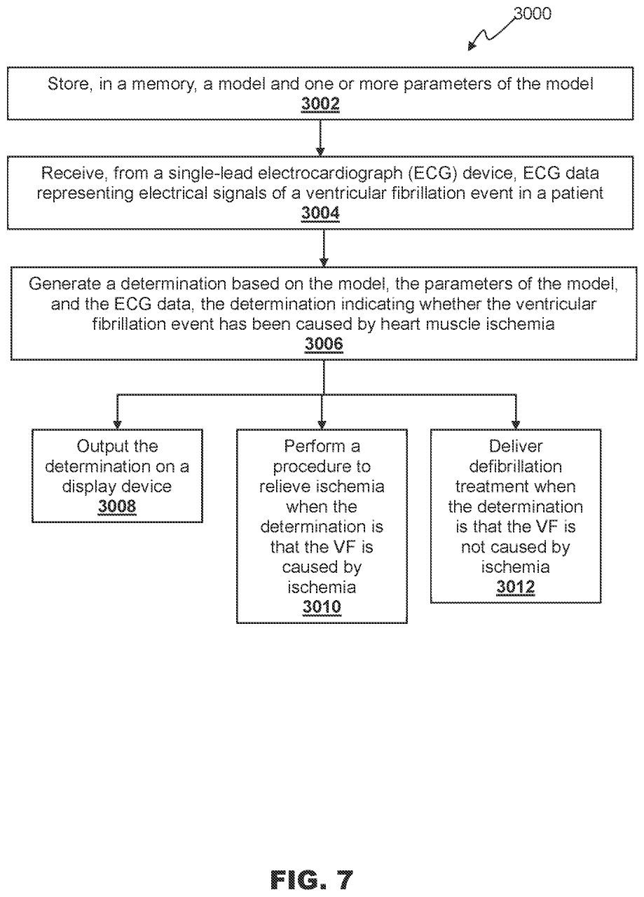

FIG. 7 is a flowchart depicting a method 3000 that can be used with embodiments of the present disclosure. At 3002, a model and associated parameters can be stored in a memory. At 3004, ECG data can be received. The ECG data can comprise single-lead electrocardiograph data. At 3006, a determination can be made based on the model, the parameters of the model, and the ECG data of the likelihood that the etiology of the fibrillation event is ischemic. At 3008, the determination can be output to a display device.

At 3010, in embodiments, one or more procedures to relieve ischemia can be performed when the determination is that the etiology is ischemic. For example, the patient can be transported to a catherization lab for bypass or other treatment. At 3012, defibrillation treatment, such as the delivery of defibrillation shocks can be performed where the determination is that the VF is not caused by ischemia.

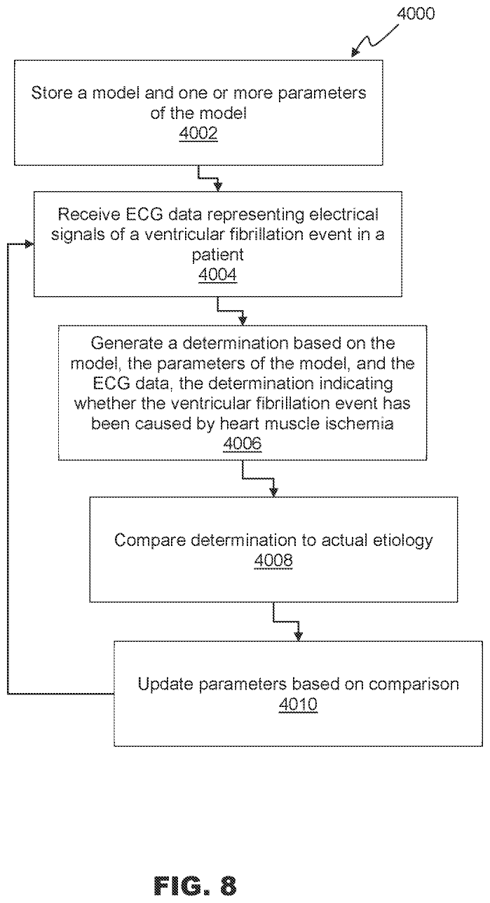

FIG. 8 is a flowchart depicting a method 4000 that can be used with embodiments of the present disclosure. As discussed above with respect to FIGS. 3 and 4 and the associated text, model 402, such as ischemia model 402a can comprise a machine-learning model, and can therefore by trained by modifying ischemia parameters 404a based on the actual etiology of input ECG data.

At 4002, a model and associated parameters can be stored in a memory. At 4004, ECG data can be received. At 4006, a determination can be made based on the model, the parameters of the model, and the ECG data of the likelihood that the etiology of the fibrillation event is ischemic. At 4008, the determination can be compared to the actual etiology. At 4010, the parameters of the model can be updated based on the comparison.

In embodiments, training of the model, by method 4000, or other methods, can occur prior to use of system 100. For example, ischemia model 402a can be trained based on prototypical or simulated data. Ischemia model 402a can also be trained during use of system 100. For example, system 100 can receive an input, either programmatically or via a user interface, indicating whether one or more determinations previously made by classifier 400 in line determinations made by medical experts having examined the patient and associated data. In this way, classifier 400 can be continually updated based on real-world situations.

In yet other embodiments, system 100 can receive updated parameters through, for example, remote software updates. As additional ischemia determinations 406a are reviewed, global updates of parameters 308 can be generated, and provided through a push or pull update of the classifier 400.

While FIG. 8 is described above with respect to ischemia classifier 400a, similar methods of calculation and comparison can be used with respect to other classifiers discussed herein, including CPP classifier 400b, cardioversion success classifier 400c, and their respective models 402 and parameters 404.

In an embodiment, classifier 400 can additionally, or in the alternative to ischemia classifier 400a, comprise coronary perfusion pressure (CPP) classifier 400b, comprising CPP model 402b which can receive ECG data 206 and produce CPP determination 404b. CPP determination 404b can be a prediction of a coronary perfusion pressure (CPP) in the subject. In embodiments, CPP determination 404b can be classification of the VF signal as falling into one of two or more classifications. For example, CPP determination 404b can indicate whether the CPP is likely to be above or below a certain threshold, such as 15 mmHg. CPP determination 404b can comprise classification among three categories, defined by threshold, such as below 10 mmHg, between 10 mmHg and 15 mmHg, and greater than 15 mmHg. As with ischemia determination 404a, CPP determination 404b can further include an indication the result was inconclusive. In yet other embodiments, CPP determination 404b can include a likelihood (such as a decimal or percentage chance) that ECG data 206 represents a signal indicative of CPP within a certain class.

CPP model 402b can comprise one or more classification algorithms that can be trained, by iterative modification of CPP parameters 404b to recognize signals that are indicative of a CPP within certain ranges.

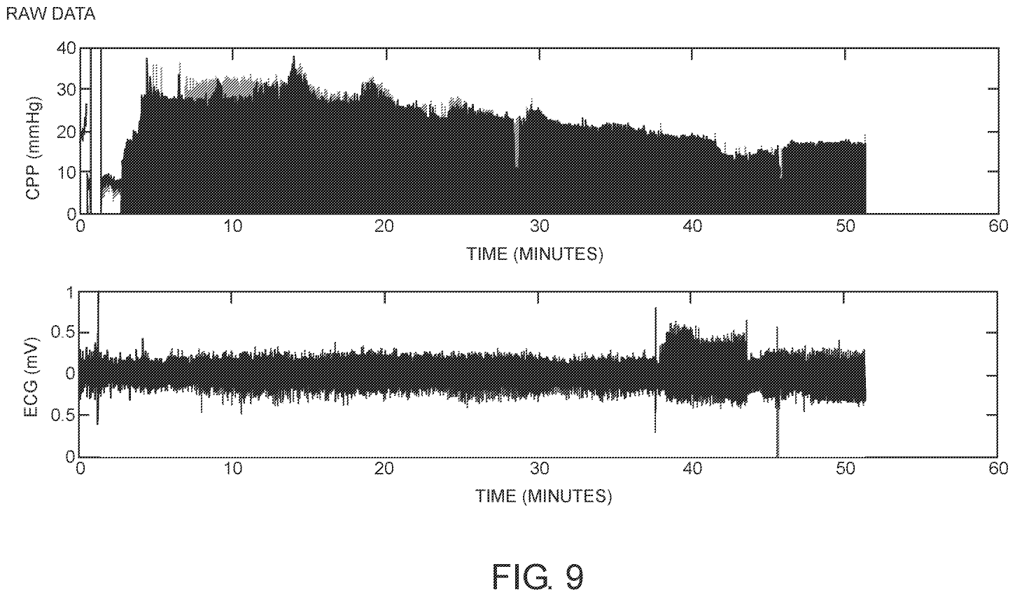

FIG. 9 is a graph depicting example ECG data and corresponding CPP data that can be provided as training inputs for embodiments of CPP model 402b The depicted example data was generated based on a porcine model of cardiac arrest. Briefly, pressure catheters were implanted in a pig and the animal was put into cardiac arrest via battery stimulation of the heart. The ECG signal (VF) was then recorded during CPR in addition to the cardiac perfusion pressure. Each model was trained with 70% a labeled data set, and the remaining test data was provided to the model.

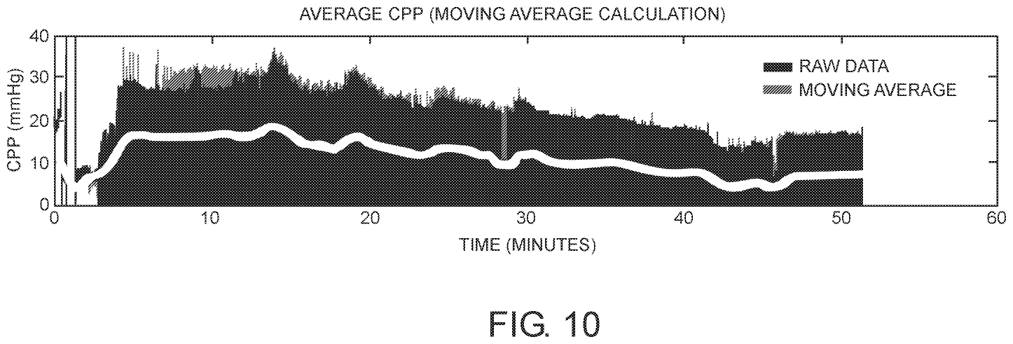

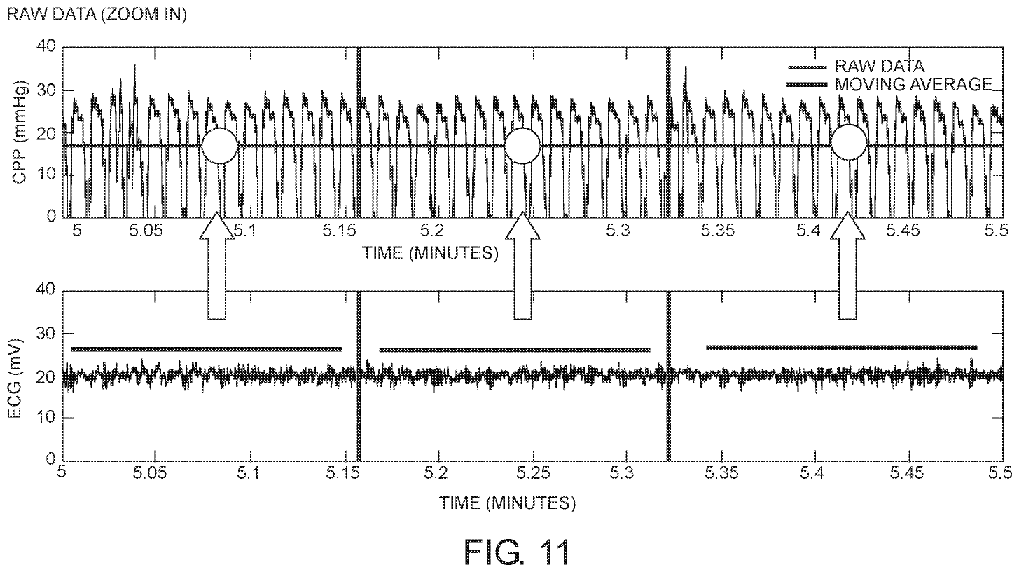

FIG. 10 is a graph depicting the moving average CPP of the CPP data depicted in FIG. 9. FIG. 11 is a view of the graph depicted in FIG. 9, zoomed in to highlight approximately 30 seconds (from a timestamp 5 minutes to a timestamp at 5.5 minutes).

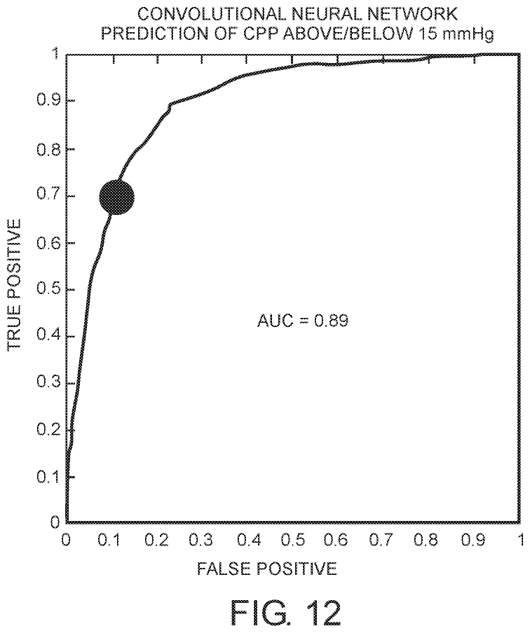

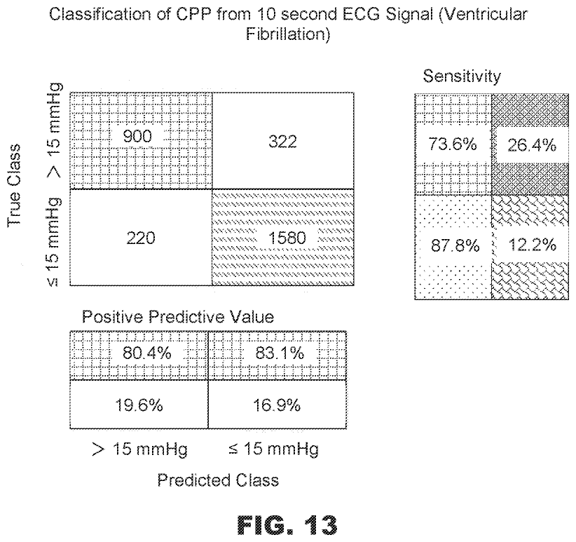

FIG. 12 is a graph depicting a receiver-operator curve resulting from testing an embodiment of system 100. Where the architecture of CPP model 404b comprised a CNN configured to predict whether the CPP was above 15 mmHg (high) or below 15 mmHg (low), the area under the curve of false positives against true positives was 0.89. FIG. 13 is a graph depicting the sensitivity and positive predictive value of the high/low classifier from a 10 second window of ECG data.

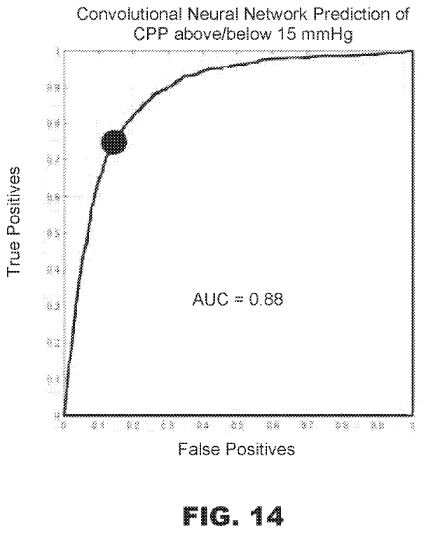

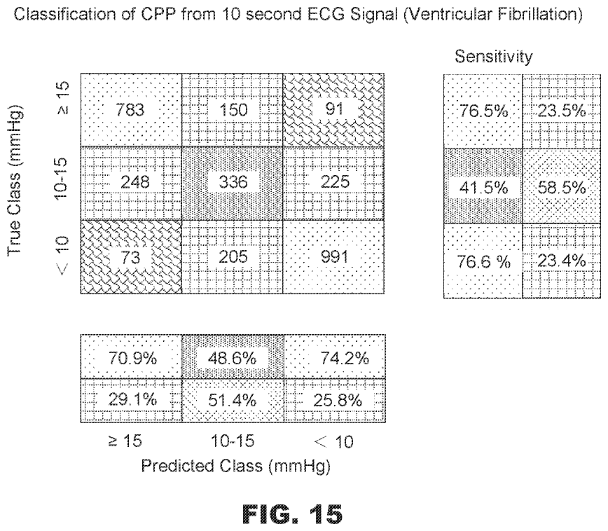

FIG. 14 is a graph depicting a receiver-operator curves resulting from testing an embodiment of system 100. Where the architecture of CPP model 402b comprised a CNN configured to predict whether the CPP was in one of three ranges: below 10 mmHg (low), between 10 mmHg and 15 mmHg (medium), or above 15 mmHg (high), the area under the curve of false positives against true positives was 0.88. FIG. 15 is a graph depicting the sensitivity and positive predictive value of the three-category classifier from a 10 second window of ECG data.

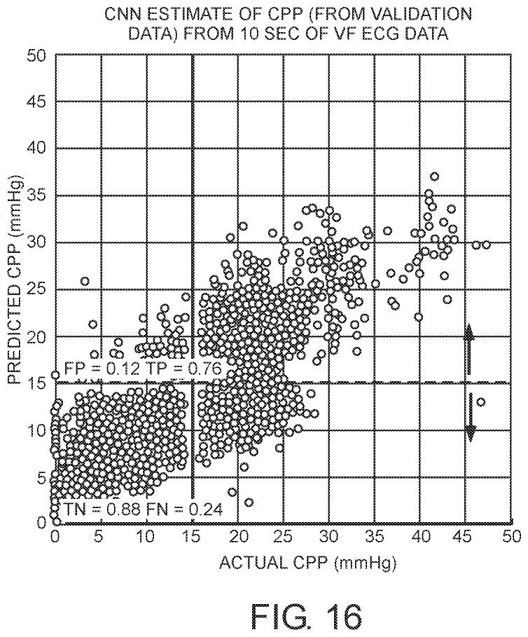

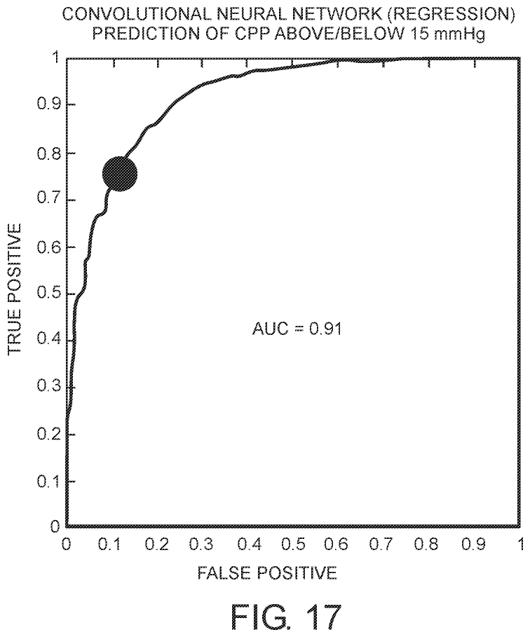

FIG. 16 is a graph depicting the actual CPP and the predicted CPP resulting from testing an embodiment of system 100. FIG. 17 is the corresponding receiver-operator curve. Where the architecture of CPP model 402b comprised a regression CNN configured to predict the CPP value (as opposed to the range), the area under the curve was 0.91.

CPP model 402b can provide a non-invasively determined prediction of CPP. This prediction can be used to optimize the delivery of CPR treatment to a subject, therefore improving outcomes and increasing the likelihood of survival. Embodiments incorporating CPP model 402b can provided to mechanical CPR controller 600, as described with respect to FIG. 6 above. Additionally, embodiments can be combined with hand pads that such as those used with automated external defibrillators that help assist a user in performing adequate chest compressions.

When manually delivered, or automated CPR compressions are guided by a feedback mechanism that optimizes cardiac perfusion pressure, instead of delivering CPR based on standard guidelines, CPR quality can be substantially improved. Previously, invasive monitoring methods were required to determine a CPP. Embodiments of the present disclosure avoid such invasive measures by inferring a CPP value from the ECG data 206.

In an embodiment, classifier 400 can additionally, or in the alternative to other classifiers discussed herein, comprise cardioversion success classifier 400c, comprising cardioversion success model 402c which can receive ECG data 206 and produce cardioversion success determination 404c. Cardioversion success determination 404c can be a prediction of the likelihood of successful cardioversion if defibrillation therapy (such as a shock from an implanted or external defibrillator) is provided during a given time window. In embodiments, cardioversion success determination 404c can be a relative value such as “low,” “medium” or “high,” or inconclusive. Cardioversion success determination can also include a probability value.

In an embodiment, cardioversion success classifier 400c can use a CPP determination 404b, such as may be provided by CPP classifier 400b or the like. For example, cardioversion success determination 400c can be “high” when the CPP determination is above a certain threshold, such as 15 mmHg.

In an alternative embodiment, cardioversion success determination model 402c can comprise a Bayesian Recurrent Neural Network (BRNN) for optimization of the timing of an attempted cardioversion. In one implementation, cardioversion success determination model 402c animals were instrumented to monitor invasive hemodynamic data. VF was induced via electrical stimulation and animals were left untouched for 3 minutes (to simulate EMS response), and at that point chest compressions were initiated. Over the course of the training, several shocks were delivered to attempt cardioversion. They were then labeled as “successful” or “unsuccessful,” based upon whether or not the VF rhythm was converted to a stable rhythm. Because the most optimal rhythm is a sinus rhythm, in instances where the post defibrillation rhythm was not a sinus rhythm, the invasive hemodynamic data was used demonstrate whether or not the rhythm could provide perfusion (i.e. atrial fibrillation, accelerated junctional, etc.). If the post-cardioversion rhythm was able to raise the CPP above 30 mmHg, it was labeled as a stable rhythm. Data from these studies were then used to train cardioversion success determination model 402c to continuously calculate a probability of successful cardioversion based upon the ECG characteristics.

Using cardioversion success classifier 400c the ECG of a patient can be continuously monitored throughout a resuscitation process. Using either technique (indirectly through estimation of CPP, or directly calculating the probability of cardioversion), CPR would then not need to be stopped, unless there was a high probability of successful cardioversion. This approach will then minimize the overall time (potentially as much as 25% when performing optimal CPR) of hypoperfusion during CPR, thereby improving patient outcomes.

Embodiments of the present disclosure can be used to determine the etiology of a cardiac arrest and provide an early diagnosis that can guide paramedics regarding treatment. For example, embodiments can enable medical personnel to rapidly determine whether the patient should be transported to a catheterization laboratory. If this can be done early in the patient's care while resuscitation is proceeding, then selection of patients who are the best candidates to go for cardiac catheterization could be accomplished more quickly and the receiving facility advised.

Embodiments can utilize short segments of ECG data from a single lead and determine the etiology of a cardiac arrest from input signal data that may appear electrically random.

Embodiments permit myocardial ischemia/infarction to be diagnosed immediately by the EMS providers placing ECG electrodes on the patient. The EMS team will then make the diagnosis of a potential blocked heart artery even while VF is present. The cardiac catheterization laboratory can be automatically alerted to the incoming patient. Further, where integrated with mechanical CPR devices, embodiments can enable CPR therapy to be performed as the patient is to the catheterization laboratory. In such circumstances, after the blockage is relieved the chances of terminating the VF will greatly increase, leading to improved patient outcomes.

The embodiments of the present disclosure can utilize computational algorithmic methods to analyze a segment (epoch) of a VF signal that otherwise appears to be a random signal offering little useful diagnostic information. Embodiments identify those patients who have suffered a cardiac arrest from a myocardial ischemia, and thereby improve patient outcomes by prompting transport to a cardiac catheterization laboratory which has already been informed that the patient may require cardiac catheterization to open a heart artery.

It should be understood that the individual steps used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

In embodiments, the various components of systems 100 and 100′ can be integrated into a single unit, or can be physically separated while remaining in electrical or communicative contact as discussed herein. Systems 100 and 100′ and/or their components or subsystems can include computing devices, microprocessors, modules and other computer or computing devices, which can be any programmable device that accepts digital data as input, is configured to process the input according to instructions or algorithms, and provides results as outputs. In one embodiment, computing and other such devices discussed herein can be, comprise, contain or be coupled to a central processing unit (CPU) configured to carry out the instructions of a computer program. Computing and other such devices discussed herein are therefore configured to perform basic arithmetical, logical, and input/output operations.

Computing and other devices discussed herein can include memory. Memory can comprise volatile or non-volatile memory as required by the coupled computing device or processor to not only provide space to execute the instructions or algorithms, but to provide the space to store the instructions themselves. In one embodiment, volatile memory can include random access memory (RAM), dynamic random access memory (DRAM), or static random access memory (SRAM), for example. In one embodiment, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the disclosure.

In one embodiment, the system or components thereof can comprise or include various modules or engines, each of which is constructed, programmed, configured, or otherwise adapted to autonomously carry out a function or set of functions. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations, and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that embodiments may comprise fewer features than illustrated in any individual embodiment described above. The embodiments described herein are not meant to be an exhaustive presentation of the ways in which the various features may be combined. Accordingly, the embodiments are not mutually exclusive combinations of features; rather, embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Moreover, reference in the specification to “one embodiment,” “an embodiment,” or “some embodiments” means that a particular feature, structure, or characteristic, described in connection with the embodiment, is included in at least one embodiment of the teaching. The appearances of the phrase “in one embodiment” in various places in the specification are not necessarily all referring to the same embodiment.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of Section 112, sixth paragraph of 35 U.S.C. are not to be invoked unless the specific terms “means for” or “step for” are recited in a claim.

The various embodiments of the present disclosure may be further understood per the included Appendices.

1. A cardiac arrest classification and treatment system comprising:
an electrocardiograph (ECG) device for receiving ECG data produced from electrical signals of a ventricular fibrillation event detected in a patient;
a processor; and
a memory storing instructions that when executed by the processor cause the processor to implement:
an ischemia classifier comprising an ischemia model and one or more parameters of the ischemia model, and configured to:
generate an ischemia determination based on the ischemia model, the parameters of the ischemia model, and the ECG data, the ischemia determination indicating whether the ventricular fibrillation event has been caused by heart muscle ischemia;
a coronary perfusion pressure (CPP) classifier comprising a CPP model and one or more parameters of the CPP model, and configured to:
generate a CPP determination based on the CPP model, the parameters of the CPP model, and the ECG data, the CPP determination indicating a predicted CPP of the patient;
a therapy determinator configured to:
direct delivery of defibrillation therapy to the patient in response to an ischemia determination indicating that the ventricular fibrillation event has been caused by heart muscle ischemia; and
direct delivery of CPR compressions to the patient based on the CPP determination.

2. The system of claim 1, further comprising a cardioversion success classifier configured to generate a likelihood of cardioversion success based on the ECG data, and wherein the therapy determinator is further configured to:
select a time window during which to direct delivery of defibrillation therapy based on the likelihood of cardioversion success;
direct a pause in the delivery of CPR compressions to the patient during the time window; and
direct the delivery of defibrillation therapy to the patient during the time window.

3. The system of claim 2 wherein the likelihood of cardioversion success is based on the CPP determination.

4. The system of claim 2 wherein the likelihood of cardioversion success is determined to be high when the CPP determination is equal to or greater than a threshold.

5. The system of claim 4 wherein the threshold is 15 mmHg.

6. The system of claim 1, further comprising an output device, and wherein directing the delivery of defibrillation therapy comprises producing an output directing a user to deliver defibrillation therapy.

7. The system of claim 1, further comprising a defibrillation device electrically coupleable to the patient for the delivery of defibrillation therapy and configured to deliver a defibrillation shock to the patient in response to the direction to delivery defibrillation therapy.

8. The system of claim 1, wherein the therapy determinator is further configured to determine a rate and pressure of CPR compressions to be delivered to the patient.

9. The system of claim 8, further comprising an output device and wherein directing the delivery of CPR compressions comprises producing an output directing a user to deliver CPR compressions at the determined rate and pressure.

10. The system of claim 8, further comprising an automated CPR module arrangeable to deliver CPR compressions to the patient at the determined rate and pressure in response to the direction to deliver CPR compressions.

11. A cardiac arrest rhythm classification and treatment system comprising:
an electrocardiograph (ECG) device for detecting electrical signals of a ventricular fibrillation event in a patient and producing ECG data;
an ischemia classifier comprising a memory and a processor, the memory storing an ischemia model and one or more parameters of the ischemia model, and instructions that when executed by the processor cause the processor to:
generate an ischemia determination based on the ischemia model, the parameters of the ischemia model, and the ECG data, the ischemia determination indicating whether the ventricular fibrillation event has been caused by heart muscle ischemia; and
a display device communicatively coupled to the ischemia classifier and configured to output the ischemia determination.

12. A cardiac arrest classification and treatment system comprising:
an electrocardiograph (ECG) device for receiving ECG data produced from electrical signals of a ventricular fibrillation event detected in a patient;
a coronary perfusion pressure (CPP) classifier comprising a memory and a processor, the memory storing a CPP model and one or more parameters of the CPP model, and instructions that when executed by the processor cause the processor to:
generate a CPP determination based on the CPP model, the parameters of the CPP model, and the ECG data, the CPP determination indicating a predicted coronary perfusion pressure of the patient; and
a display device communicatively coupled to the classifier and configured to output the CPP determination.

13. A cardiac arrest classification and treatment system comprising:
an electrocardiograph (ECG) device for detecting electrical signals of a ventricular fibrillation event during which a patient is receiving cardiopulmonary resuscitation (CPR) compressions and producing ECG data;
a cardioversion success classifier configured to generate a likelihood of cardioversion success based on the ECG data; and
and a therapy determinator configured to:
select a time window during which to direct delivery of defibrillation therapy based on the likelihood of cardioversion success,
direct a pause in the delivery of CPR compressions to the patient during the time window, and
direct delivery of defibrillation therapy to the patient during the time window.

14. The system of claim 13, wherein the cardioversion success classifier is configured to generate the likelihood of cardioversion success by calculating a predicted coronary perfusion pressure of the patient and determining that the likelihood of cardioversion success is high when the predicted coronary perfusion pressure of the patient is equal to or greater than a threshold.

15. The system of claim 11, further comprising an automated external defibrillation module.

16. The system of claim 11, further comprising an automated cardiopulmonary resuscitation module.

17. The system of claim 1, wherein the electrical signals of the ventricular fibrillation event comprise a short epoch.

18. The system of claim 17, wherein the short epoch has a length between 2 seconds and 30 seconds.

19. The system of claim 18, wherein the short epoch has a length of 10 seconds.

20. A computer-implemented method for classifying the etiology of a cardiac arrest comprising:
storing, in a memory, an ischemia model and one or more parameters of the ischemia model;
receiving, from an electrocardiograph (ECG) device, ECG data representing electrical signals of a ventricular fibrillation event in a patient; and
generating an ischemia determination based on the ischemia model, the parameters of the ischemia model, and the ECG data, the ischemia determination indicating whether the ventricular fibrillation event has been caused by heart muscle ischemia.

21. The method of claim 20, further comprising performing a procedure on the patient to relieve heart muscle ischemia when the ischemia determination is that the ventricular fibrillation event has been caused by heart muscle ischemia.

22. A computer-implemented method for optimizing the delivery of cardiopulmonary respiration (CPR) to a patient comprising:
storing, in a memory communicatively couplable to a processor, a coronary perfusion pressure (CPP) model and one or more parameters of the CPP model;
receiving, from an electrocardiograph (ECG) device, ECG data representing electrical signals of a ventricular fibrillation event in a patient;
generating, by the processor, a determination based on the CPP model, the parameters of the CPP model, and the ECG data, the determination indicating a predicted coronary perfusion pressure of the patient; and
delivering one or more CPR compressions to the patient, the timing and pressure of the CPR determined at least partially based on the predicted coronary perfusion pressure.

23. A computer-implemented method monitoring and classifying a cardiac arrest rhythm comprising:
storing, in a memory communicatively couplable to a processor, an ischemia model, parameters of the ischemia model, a coronary perfusion pressure (CPP) model and parameters of the CPP model;
receiving, by the processor, electrocardiograph (ECG) data produced from electrical signals of a ventricular fibrillation event detected in a patient;
generating, by the processor, an ischemia determination based on the ischemia model, the parameters of the ischemia model, and the ECG data, the ischemia determination indicating whether the ventricular fibrillation event has been caused by heart muscle ischemia;
generating, by the processor, a CPP determination based on the CPP model, the parameters of the CPP model, and the ECG data, the CPP determination indicating a predicted CPP of the patient;
directing delivery of defibrillation therapy to the patient in response to an ischemia determination indicating that the ventricular fibrillation event has been caused by heart muscle ischemia; and
directing delivery of CPR compressions to the patient based on the CPP determination.

24. The method of claim 23 further comprising determining a rate and pressure of CPR compressions to be delivered to the patient.

25. The method of claim 23 further comprising:
generating a likelihood of cardioversion success based on at least one of the ECG data and the CPP determination;
selecting a time window during which to direct delivery of defibrillation therapy based on the likelihood of cardioversion success;
directing a pause in the delivery of CPR compressions to the patient during the time window; and
directing the delivery of defibrillation therapy to the patient during the time window.
